(19) United States
(12) Patent Application Publication
(10) Pub. No.: US 2026/0112504 A1
(43) Pub. Date: Apr. 23, 2026

(54) METHODS, DEVICES, AND SYSTEMS FOR ESTIMATION OF BIOLOGICAL AGE

(71) Applicants: Yuanxu GAO, Weifang (CN); Kang ZHANG, Macao (CN)

(72) Inventors: Yuanxu GAO, Weifang (CN); Kang ZHANG, Macao (CN)

(21) Appl. No.: 19/414,736

(22) Filed: Dec. 10, 2025

Related U.S. Application Data
(63) Continuation of application No. PCT/CN2023/099561, filed on Jun. 10, 2023.

(51) Int. Cl. G16H 50/30; G06N 3/045; G16H 30/40

(52) U.S. Cl. CPC G16H 50/30; G06N 3/045; G16H 30/40

(57) ABSTRACT

Provided herein in some embodiments are methods, devices, storage media, and systems using a model having a multi-modal transformer-based architecture with cross-attention which combines facial, tongue and retina images to estimate biological age (BA). The difference between chronological age (CA) and BA (AgeDiff) can be used as a standalone biomarker, or conjunctively alongside other known factors for risk stratification and progression prediction of chronic diseases.

### CROSS-REFERENCE TO RELATED APPLICATION

This application is a continuation of International Patent Application No. PCT/CN2023/099561, filed on Jun. 10, 2023, entitled “ACCURATE ESTIMATION OF BIOLOGICAL AGE USING A TRANSFORMER-BASED HOLISTIC REPRESENTATION OF MULTI-MODAL IMAGE INFORMATION,” which application is herein incorporated by reference in its entirety for all purposes.

### FIELD

The present disclosure relates in some aspects to methods, devices, storage media, and systems involving unified processing of multimodal input for accurate estimation of biological age, including in some aspects using transformer-based holistic representation of multi-modal image information.

### BRIEF SUMMARY

The aging process is inevitable and is a risk factor for chronic diseases. The biological age (BA) of each individual contains structural and functional determinants of aging, and its difference (AgeDiff) from the chronological age (CA) can be used as a biomarker for accelerated aging caused by underlying pathologies. Described herein is a multi-modal Transformer-based architecture which can estimate BA based on facial, fundus and retina images. The results demonstrated that BA of healthy individuals can be accurately estimated. Significant deviations of AgeDiff are present in individuals with chronic diseases, and AgeDiff can be used to accurately detect systematic diseases and identify progression risks. The present disclosure teaches a method to use easily and readily acquired patient data to identify chronic diseases.

In some embodiments, provided herein is a method of training a model for biological age estimation, wherein the model comprises a first projection module, a second projection module, a third projection module, and a multimodal transformer comprising a cross-attention module, the method comprising: (a) obtaining multimodal input data of a subject, wherein the multimodal input data comprise data in a first modality, data in a second modality, and data in a third modality: (b) passing the data in a first modality, the data in a second modality, and the data in a third modality to the first projection module, the second projection module, and the third projection module, respectively, to construct the corresponding image tokens and classification tokens: (c) passing the image tokens and classification tokens to the multimodal transformer comprising the cross-attention module, wherein the cross-attention module comprises three branches, wherein each branch processes image tokens of one of the three modalities, and wherein the cross-attention module fuses the image tokens and the classification tokens using cross-attention fusion comprising fusing a classification token from one of three modalities and image tokens from the other two modalities; and (d) passing an output of the multimodal transformer to a plurality of multilayer perceptrons for biological age estimation, thereby training the model for providing biological age estimation.

In some embodiments, provided herein is a method of training a model for biological age estimation, wherein the model comprises a first projection module, a second projection module, a third projection module, and a multimodal transformer comprising a cross-attention module, the method comprising: (a) obtaining multimodal input data of a subject, wherein the multimodal input data comprise three image modalities: retinal images, tongue images, and facial images: (b) passing the retinal images, tongue images, and facial images to the first projection module, the second projection module, and the third projection module, respectively, to construct the corresponding image tokens and classification tokens: (c) passing the image tokens and classification tokens to the multimodal transformer comprising the cross-attention module, wherein the cross-attention module comprises three branches that process image tokens of the retinal images, the tongue images, and facial images, respectively, and wherein the cross-attention module fuses the image tokens and the classification tokens using cross-attention fusion comprising fusing classification tokens from one of the three image modalities and image tokens from the other two image modalities: (d) passing an output of the multimodal transformer to a plurality of multilayer perceptrons for biological age estimation, thereby training the model for providing biological age estimation; and (e) obtaining the difference AgeDiff between the estimated biological age (BA) of an individual and the individual's chronological age (CA), wherein AgeDiff=|BA−CA|.

In some embodiments, provided herein is a method of estimating a biological age for a subject, comprising: receiving a plurality of images of the subject and a set of text data associated with the subject; and generating a plurality of tokens by: converting the plurality of images into a plurality of visual tokens; and converting the set of text data into one or more textual token: estimating the biological age of the subject by inputting the plurality of tokens into a trained machine learning model comprising a plurality of cross-attention modules with intramodal and intermodal attention.

In some embodiments, the plurality of images can comprise: one or more tongue images, one or more facial images, one or more fundus images, or any combination thereof.

In any of the embodiments herein including any preceding embodiment, the set of text data can comprise narrative text, one or more text-field data, or a combination thereof.

In any of the embodiments herein including any preceding embodiment, the disclosed methods can further comprise providing a diagnosis for the subject based on the estimated biological age of the subject.

In some embodiments, the diagnosis can comprise: an identification of a disease, a prediction of a progression of the disease, a risk factor associated with the disease, or any combination thereof.

In any of the embodiments herein including any preceding embodiment, the disclosed methods can further comprise providing an output indicative of at least a portion of the plurality of images as contributing to the estimated biological age.

In any of the embodiments herein including any preceding embodiment, the method can be a computer-implemented method.

In some embodiments, provided herein is a system comprising: at least one hardware processor; and one or more software modules configured to, when executed by the at least one hardware processor, perform the method of any embodiment disclosed herein including any preceding embodiment.

In some embodiments, provided herein is a non-transitory computer-readable medium having instructions stored thereon, wherein the instructions, when executed by a processor, cause the processor to perform the method of any embodiment disclosed herein including any preceding embodiment.

In some embodiments, provided herein is a system comprising: at least one hardware processor: non-transitory computer-readable medium coupled to at least one hardware processor, optionally wherein the coupling is over a network; and instructions stored in the non-transitory computer-readable medium, wherein the instructions when implemented by the processor, configure the system to perform the method of any embodiment disclosed herein including any preceding embodiment.

### BRIEF DESCRIPTION OF THE DRAWINGS

The drawings illustrate certain features and advantages of this disclosure. These embodiments are not intended to limit the scope of the appended claims in any manner. Various aspects of the disclosed methods, devices, and systems are set forth with particularity in the appended claims, and an understanding of the features and advantages of the disclosed methods, devices, and systems will be obtained by reference to the following detailed description of illustrative embodiments and the accompanying drawings, of which:

FIG. 1A describes an exemplary AI system for BA estimation using a combination of retinal images, tongue images and facial images, BA-based models for detection and progression prediction of systemic diseases, and relationship analysis between BA and known risk factors.

FIG. 1B shows a transformer-based architecture with a cross-attention module for BA estimation using a combination of retinal images, tongue images and facial images. The networks are optimized using the loss between real age and BA with BP algorithm. A cross-attention module which consists of a stack of Z multi-modal Transformer encoders. Each uses three different branches to process image tokens of different modalities and fuse the tokens at the end by an efficient module based on cross attention of the CLS tokens (type (d) in FIG. 1C). FIG. 1C shows comparison of four types of multi-modal fusion implementations. (a) All-attention fusion where all tokens are bundled together without considering any characteristic of tokens. (b) Class token fusion, where only CLS tokens are fused as it can be considered as global representation of one branch. (c) Pairwise fusion, where tokens at the corresponding spatial locations are fused together and CLS are fused separately. (d) Cross-attention, where CLS token from one branch and patch tokens from another branch are fused together.

FIGS. 2A-2B show exemplary data depicting the impact of chronic diseases and environmental factors on BA in both the internal and external cohorts. Correlation analysis of the predicted BA versus CA generated using the multi-modal-fusion architecture on the internal test set and external test set.

FIG. 3 describes exemplary data depicting the performance of the AI models in the identification of six common chronic systematic diseases on internal test set. ROC curves represent the risk-factor-only model, the multi-modal-fusion model and the combined model.

FIG. 4 describes exemplary data depicting Kaplan-Meier plots for the prediction of six common chronic systematic diseases on internal test set. The y axis is the survival probability, measuring the probability of not progressing to a disease outcome. The x axis is the time in months. Survival curves in different colors represent the high-risk and low-risk subgroups stratified by the upper quartiles in the tuning dataset. Shaded areas are 95% CI.

FIG. 5 describes exemplary data depicting Grad-CAM++ results on three-modality inputs of one participant on internal training set at [100, 250, 300, 350, 400, 450, 500] training epochs. The saliency maps gradually provide visual clues in the training process where the network is optimized with the loss between BA and CA.

FIG. 6 describes exemplary data depicting a comparison of AgeDiff for individuals who are healthy or have any disease, for each decade of life. Statistics compare AgeDiff of the two groups using Student's t-test (p<0.05 for all groups). Distribution of AgeDiff of individuals above the age of 60 (HC: Healthy control: AD: Any disease) is shown. Calculation of AgeDiff is based on the difference between an individual's predicted BA and actual CA. Sex distribution for individuals of increased age (BA>CA) in each group is also shown.

FIG. 7 describes exemplary data depicting Bland-Altman plots for the agreement between the predicted BA and CA on internal test set and external test set (up: face, tongue, funuds and fusion from left to right) and external test set (bottom: face, tongue, funuds and fusion from left to right). The x axis represents the mean of predicted BA and CA, and the y axis represents the difference between the two measurements.

FIG. 8 describes exemplary data depicting the top 13 variants in terms of attributable AgeDiff using SHAP method (left and middle). Top 13 variants in terms of attributable AgeDiff and HRs for Any Disease (right). Estimates are based on internal test set. Error bars denote 95% CI.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

Aging is a risk factor for many chronic diseases. However, the identification of suitable predictors of universal aging for use in health management and clinical practice has been difficult [1]. This is likely due to the heterogeneous nature of the underlying tissues and organ vulnerabilities associated with aging that is not simply restricted to the passage of time. Biological age (BA) on the other hand takes into account the impact of structural and functional changes that contribute to aging [2]. These could be influenced by genetic and/or environmental factors. Thus, the ability to quantify BA may be clinically important to identify patients at-risk for age-related diseases and raises the possibility for early intervention. Artificial intelligence (AI) approaches have been developed to predict BA from a number of biomarkers of aging, such as leukocyte telomere length [3], DNA methylation-based epigenetic clock [4], brain image-derived brain age [5], [6], retinal age [7], [8] and facial age [9], [10]. The retina in particular has been recognized as a window to the brain due to the presence of central nervous system derived axons in the optic nerve, as well as similarities in the expression of cytokines and immune modulators [11]. The retinal age gap, the difference in the predicted retinal age and the chronological age (CA), has been used to assess brain health [12], [13]. Facial age has also emerged as a potential predictor for skin health [9], [10]. However, while estimation of the BA of specific organs or systems may be useful to derive information regarding organ-specific diseases, utilization of BA to its full potential will undoubtedly need to take into account the heterogeneous nature of aging. The modelling of the impact of chronic diseases, such as coronary heart disease (CHD), cardiovascular disease (CVD), chronic kidney disease (CKD), diabetes, hypertension and stroke will require integrated information from multiple systems.

A multi-modal image fusion AI model of retinal fundus, facial and tongue images was applied to predict BA capable of reflecting the physiological or pathophysiological state in multiple organ systems. Tongue images may be a potential indicator for microbiome exposure and may reflect the state of oral and gastrointestinal track health [14], [15]. This AI prediction model can be optimized by exploiting image detail using a joint loss function to represent the progressive nature of aging and to tolerate minor errors in modeling. The AI model was trained and validated using fundus, facial and tongue images from healthy participants, and employed the model to estimate the impact of diseases and lifestyle factors on BA using images from participants with a number of chronic diseases and/or known risk factors for the development of chronic diseases. Multi-modal BA output is the closest to the true age in the healthy populations. BA is markedly increased in various diseases and unhealthy lifestyle habits and is a strong predictor of chronic diseases.

The methods disclosed herein propose a multi-modal fusion framework that incorporates facial, tongue and retina image detail enhancement and a joint loss function for BA prediction. The model was validated using an independent dataset and demonstrated robustness, the ability to reflect the progressive nature of aging, and improved predictive accuracy compared to the recently reported approaches for BA prediction using retinal age [7]. While previous studies have demonstrated facial or retinal age to be a biomarker of aging [7], [9], [10], the study expanded this potential by combining and integrating retinal, tongue and facial images to gain a more complete portrait of BA. The AI model achieved comparable BA prediction on retinal age to previous studies (around 2.5 years versus CA). However, when combined with facial and tongue images, the multi-modal AI achieved BA predictions within 2 years for healthy individuals. This is the most accurate phenotypic BA prediction to the knowledge [20]-[22]. It is superior to established BA prediction models such as DNA methylation clocks [4], [23], transcriptome aging clocks [22], [24] and blood profiles [25], [26]. The AI model also shows statistically significant differences in BA between healthy and diseased subjects, indicating that the impact of diseases in BA and the potential of the BA as a novel effective biomarker of aging and age-related disease research. The study showed a link between accelerated BA and risk of chronic diseases such as CHD, CVD, CKD, stroke, hypertension, and diabetes.

Prediction of tissue and organ age is currently exemplified by retinal age, which is able to correlate between retinal neuronal and vascular changes and age-related brain diseases [11], [27]. This raises the possibility of using retinal age as a surrogate measure of brain and vascular BA. The retina and cerebrum do share high similarities in microvasculature [28] and aging outcomes, such as the accumulation of mitochondria oxidative stress [29]. However, BA predictions based on single organ systems, while useful to offer insight into system-specific diseases, does not offer a sufficiently accurate prediction of the overall physiological or pathophysiological state of the individual. Facial and tongue images may therefore add other dimensions to accurately estimate BA. Several population-based studies [9], [30], [31] have shown that aging concomitantly alters the retina, brain, skin and the gastrointestinal tract. Indeed, it is possible that tongue health may offer a window into gastrointestinal tract status and also microbiome exposure [14], [15]. Facial images may offer an assessment of direct sun and air exposure. These links will require further investigation, and will undoubtedly uncover interesting, and important relationships between chronic diseases and tongue and facial features. Nevertheless, the results showing that the predicted BA using fundus images can be improved by incorporating facial and tongue images supports the argument that tongue and facial images, when combined with AI, may offer insights into an individual's overall physiology.

In some embodiments, disclosed herein is a method of using a multi-modal image-based AI prediction as a large-scale screening tool for individuals at high risk for various chronic diseases. The BA predictions based on the model offer unique advantages of detecting the risk, as well as prognosis, of a range of diseases through a fast, non-invasive and economical method. Additionally, these predictions can be made even more accessible by incorporating smartphone-based teleophthalmology and facial and tongue imaging assessment [32]. There have been ethical and privacy concerns with using facial images for BA prediction. However, these concerns will be somewhat mitigated with the fusion approach since the facial images are combined with fundus and retina images for analyses. In conclusion, the methods disclosed herein revealed the potential utility of using multi-modal images to predict BA, which can be used to identify individuals at risk of developing chronic diseases and to intervene so the disease risks can be reduced.

Exemplary embodiments provided herein include:

Embodiment 1. A method of training a model for biological age estimation, wherein the model comprises a first projection module, a second projection module, a third projection module, and a multimodal transformer comprising a cross-attention module, the method comprising: (a) obtaining multimodal input data of a subject, wherein the multimodal input data comprise data in a first modality, data in a second modality, and data in a third modality: (b) passing the data in a first modality, the data in a second modality, and the data in a third modality to the first projection module, the second projection module, and the third projection module, respectively, to construct the corresponding image tokens and classification tokens: (c) passing the image tokens and classification tokens to the multimodal transformer comprising the cross-attention module, wherein the cross-attention module comprises three branches, wherein each branch processes image tokens of one of the three modalities, and wherein the cross-attention module fuses the image tokens and the classification tokens using cross-attention fusion comprising fusing a classification token from one of three modalities and image tokens from the other two modalities; and (d) passing an output of the multimodal transformer to a plurality of multilayer perceptrons for biological age estimation, thereby training the model for providing biological age estimation.

Embodiment 2. The method of Embodiment 1, wherein each of the first projection module, the second projection module, and the third projection module is independently a linear projection module.

Embodiment 3. The method of Embodiment 1 or Embodiment 2, wherein the first projection module, the second projection module, and the third projection module are linear projection modules.

Embodiment 4. The method of any one of Embodiments 1-3, wherein the multimodal transformer comprises a first Swin-Transformer encoder for the image tokens and classification tokens from the data in the first modality, a second Swin-Transformer encoder for the image tokens and classification tokens from the data in the second modality, and a third Swin-Transformer encoder for the image tokens and classification tokens from the data in the third modality.

Embodiment 5. The method of any one of Embodiments 1-4, wherein the multimodal transformer comprises Z-stack encoders each having a cross-attention module.

Embodiment 6. The method of Embodiment 5, wherein the cross-attention module in each stack comprises three branches, each of which is configured to process image tokens of one of the three modalities.

Embodiment 7. The method of any one of Embodiments 1-6, wherein the first modality, the second modality, and the third modality are medical image modalities.

Embodiment 8. The method of any one of Embodiments 1-7, wherein the first modality, the second modality, and the third modality are retinal images, tongue images, and facial images, respectively.

Embodiment 9. The method of Embodiment 8, wherein the retinal images are fundus images.

Embodiment 10. The method of Embodiment 8 or Embodiment 9, wherein the facial images are 3D facial stereophotogrammetry images.

Embodiment 11. The method of any one of Embodiments 1-10, further comprising obtaining the difference AgeDiff between an estimated biological age (BA) of an individual and the individual's chronological age (CA), wherein AgeDiff=|BA−CA|.

Embodiment 12. A method of for biological age estimation in an individual, the method comprising: receiving a prompt for obtaining an estimated biological age and data in the first modality; data in the second modality, and data in the third modality of the individual, and generating the estimated biological age by inputting the prompt and the data in the three modalities in a trained model generated by the method of any one of Embodiments 1-11.

Embodiment 13. The method of Embodiment 12, further comprising obtaining the difference AgeDiff between the estimated biological age (BA) of the individual and the individual's chronological age (CA), wherein AgeDiff=|BA−CA|.

Embodiment 14. The method of Embodiment 13, comprising using AgeDiff to predict a 5-year risk of the individual developing a chronic disease.

Embodiment 15. The method of Embodiment 13, comprising using a combination of AgeDiff and one or more known risk factors for a chronic disease to predict a 5-year risk of the individual developing the chronic disease.

Embodiment 16. The method of Embodiment 14 or Embodiment 15, wherein the chronic disease is coronary heart disease (CHD), cardiovascular disease (CVD), chronic kidney disease (CKD), stroke, hypertension, or diabetes.

Embodiment 17. A method of training a model for biological age estimation, wherein the model comprises a first projection module, a second projection module, a third projection module, and a multimodal transformer comprising a cross-attention module, the method comprising: (a) obtaining multimodal input data of a subject, wherein the multimodal input data comprise three image modalities: retinal images, tongue images, and facial images: (b) passing the retinal images, tongue images, and facial images to the first projection module, the second projection module, and the third projection module, respectively, to construct the corresponding image tokens and classification tokens: (c) passing the image tokens and classification tokens to the multimodal transformer comprising the cross-attention module, wherein the cross-attention module comprises three branches that process image tokens of the retinal images, the tongue images, and facial images, respectively, and wherein the cross-attention module fuses the image tokens and the classification tokens using cross-attention fusion comprising fusing classification tokens from one of the three image modalities and image tokens from the other two image modalities: (d) passing an output of the multimodal transformer to a plurality of multilayer perceptrons for biological age estimation, thereby training the model for providing biological age estimation; and (e) obtaining the difference AgeDiff between the estimated biological age (BA) of an individual and the individual's chronological age (CA), wherein AgeDiff=|BA−CA|.

Embodiment 18. A system comprising: at least one hardware processor; and one or more software modules configured to, when executed by the at least one hardware processor, perform the method of any one of Embodiments 1-17.

Embodiment 19. A non-transitory computer-readable medium having instructions stored thereon, wherein the instructions, when executed by a processor, cause the processor to perform the method of any one of Embodiments 1-17.

Embodiment 20. A system comprising: at least one hardware processor: non-transitory computer-readable medium coupled to at least one hardware processor, optionally wherein the coupling is over a network; and instructions stored in the non-transitory computer-readable medium, wherein the instructions when implemented by the processor, configure the system to perform the method of any one of Embodiments 1-17.

### EXAMPLES

The following examples are included for illustrative purposes only and are not intended to limit the scope of the present disclosure.

Aging in an individual refers to the temporal change, mostly decline, in the body's ability to meet physiological demands. Biological age (BA) is a biomarker of chronological aging, and can be used to stratify populations to predict certain age related chronic diseases. BA can be predicted from biomedical features such as brain MRI, retina or facial images, but the inherent heterogeneity in the aging process limits the usefulness of BA predicted from individual body systems. The methods disclosed herein teach a multi-modal Transformer-based architecture with cross-attention which was able to combine facial, tongue and retina images to estimate BA. The model was trained using facial, tongue and retina images from 11,223 healthy subjects, and demonstrated that using a fusion of the three image modalities achieved the most accurate BA predictions. The approach was validated on a test population of 2,840 individuals with six chronic diseases and obtained significant difference between chronological age (CA) and BA (AgeDiff) than that of healthy subjects. AgeDiff has the potential to be utilized as a standalone biomarker, or conjunctively alongside other known factors for risk stratification and progression prediction of chronic diseases. The results therefore highlight the feasibility of using multi-modal images to estimate and interrogate the aging process.

### Example 1—Overview of the Model

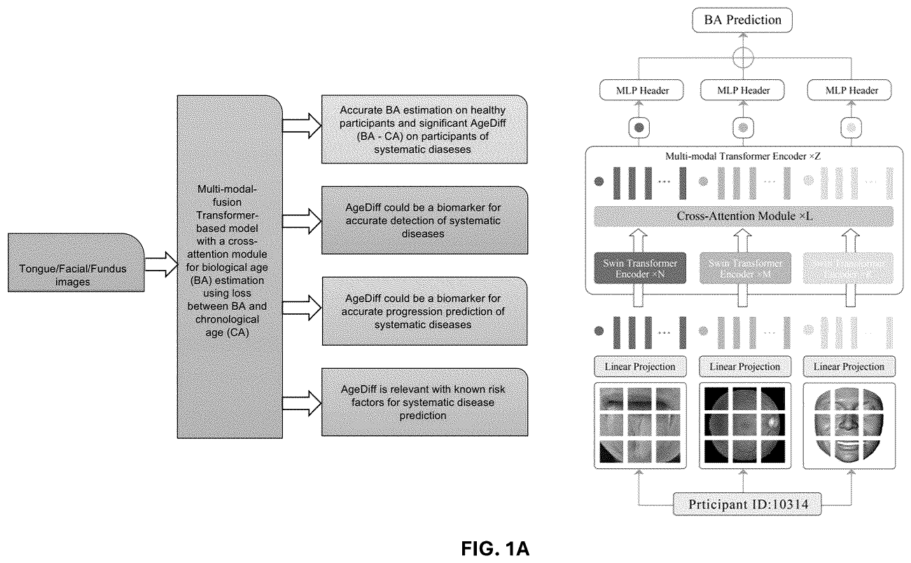

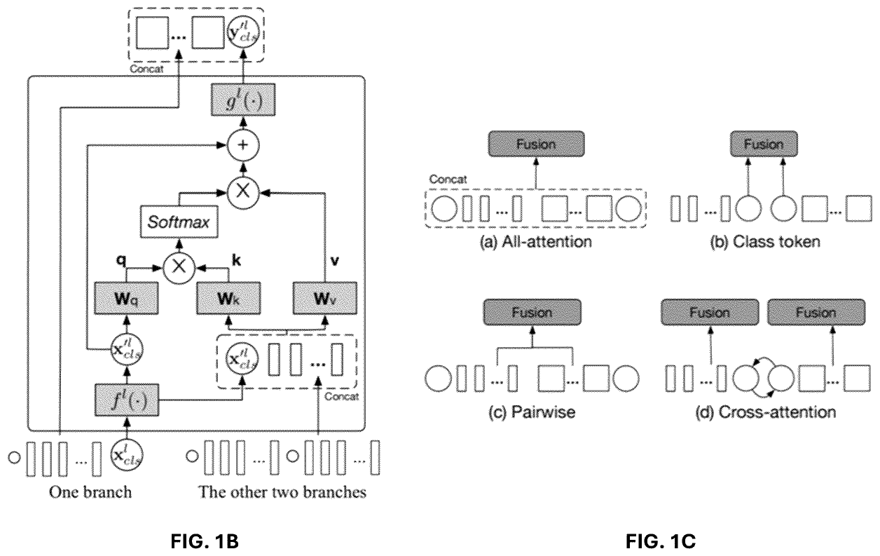

An overview of the study incorporating the AI model is shown in FIG. 1A. The AI model is a Transformer-based architecture which incorporates a cross-attention module for BA estimation using a combination of fundus, facial and tongue images. The input images of three modalities are first sent to three linear projection modules to construct the corresponding image tokens and CLS tokens. These ViT-like tokens are regarded as the input of a Multi-modal Transformer (MMT) that contains Z-stack encoders with a cross-attention module (CAM). Each CAM uses three branches to process image tokens of three modalities and fuses the tokens at the end based on the CLS tokens of CAM. A cross-attention fusion (FIG. 1B) strategy, which involves the CLS token of one modality and image tokens of the other two modalities, is used in the model and demonstrates advantages over other heuristic approaches (FIG. 1C). The outputs of the MMT encoders are linked to standard MLP headers for BA prediction. The whole architecture is optimized using the loss function between the CA and the predicted BA using a back-propagation algorithm.

### Example 2—Patient Characteristics

The general scheme of the study design and procedures are described in FIG. 1A. The training dataset contains subjects in the northern China cohort who were followed longitudinally for regular health checks starting with a cross-sectional study. A total of 14,063 subjects consented to participate in the study. They were subjected to 3D face, tongue and retina scanning and relevant metadata were extracted from their medical records. Blood was drawn after fasting followed by medical follow-up. The metadata included demographic information, life-style (including smoking, alcohol use) and outcomes from routine physical examinations and clinical laboratory assays (FIG. 1A, Table 1). All participants from the discovery cohort were split into mutually exclusive sets for training, tuning and internal validation of the AI algorithm at an 80%: 10%: 10% ratio. The southern China cohort of 2,766 subjects serves as an independent validation cohort.

### Example 3—BA Estimation by Facial, Tongue and Retinal Images

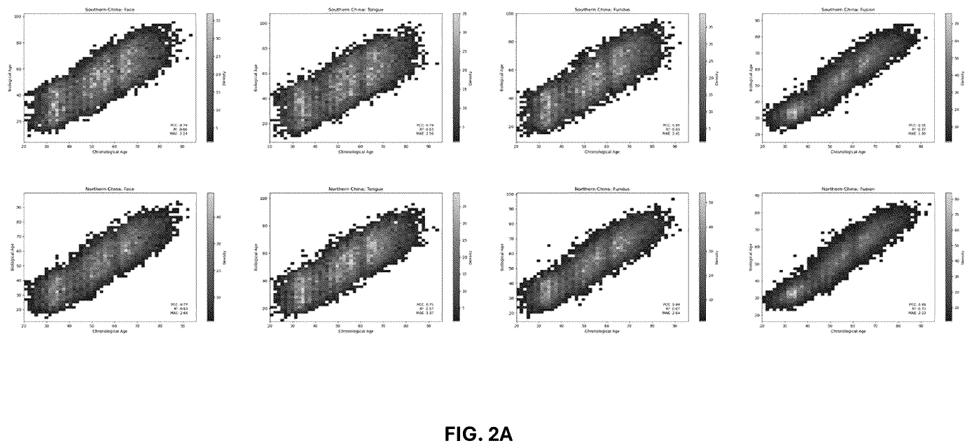

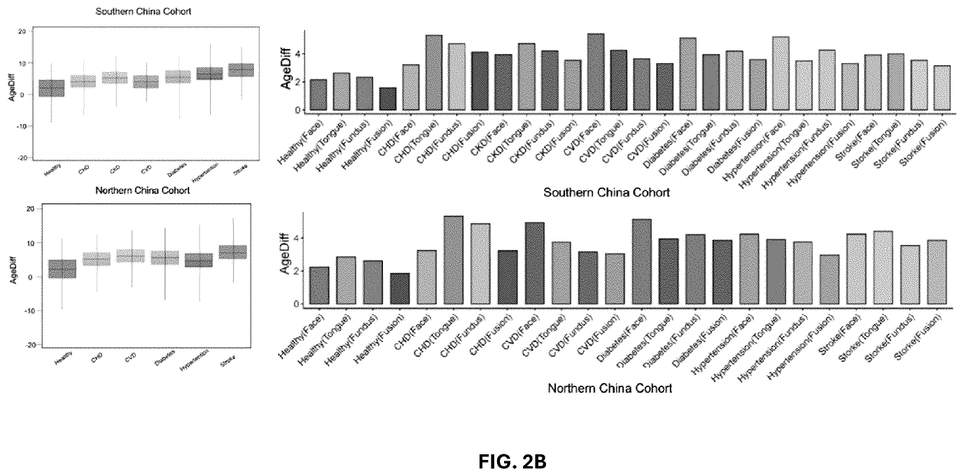

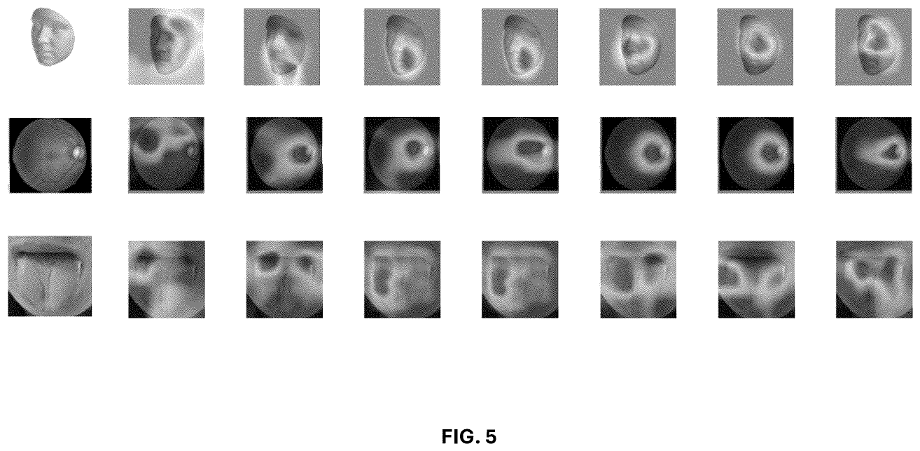

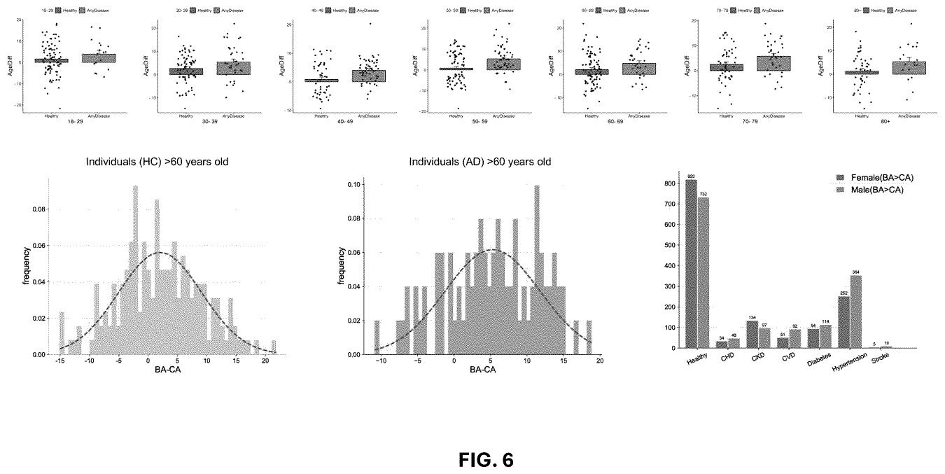

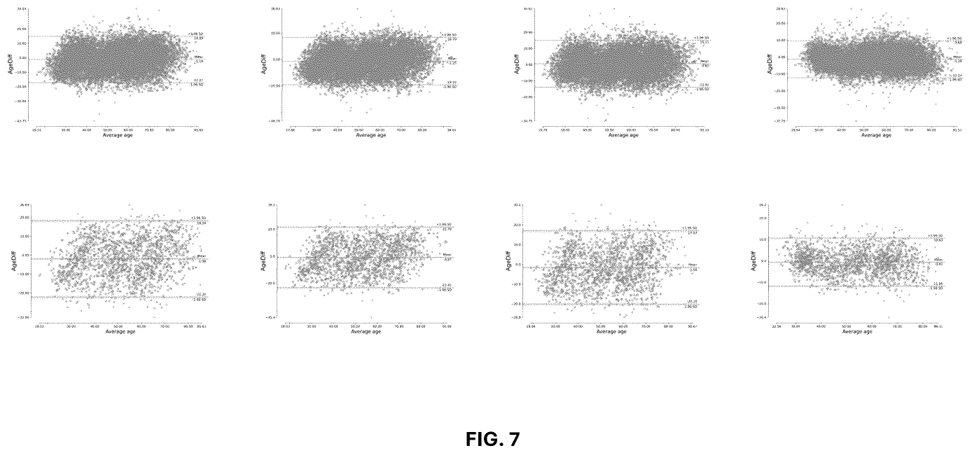

A multi-modal image fusion approach, using fundus, tongue and facial images, was applied in the AI model to estimate BA. The AI model was trained using images from healthy participants to predict BA. The accuracy of the AI model-predicted BA was determined by its difference from the CA of the corresponding participant using healthy participants. The scatter plots of BA predictions from the test sets in internal and external cohorts are shown in FIGS. 2A-2B. In both cohorts, BA predictions using the multi-modal image fusion approach produced a better correlation with the CA (Pearson's correlation coefficient (PCC) of 0.91 in the internal cohort and PCC of 0.88 in the external cohort). The mean absolute error (MAE) as well as the Coefficient of determination R² were also improved. Using Grad-CAM++ as an interpretation for the AI findings, the multi-modal-fusion AI model paid more attention to regions near the lip and center in tongue image, vascular-density region in retinal fundus image and eve region in facial image (FIG. 5). The data therefore indicate that the multi-modal image fusion AI model was able to accurately predict BA and was superior to BA prediction using either of the three image modalities alone.

The multi-modal image fusion AI model was then used to evaluate the impact of chronic diseases and environmental factors on BA in both the internal and external cohorts (FIGS. 2A-2B). The BA of each subject was predicted and the AgeDiff was evaluated, as above. The mean AgeDiff was plotted and it was found that in individuals with chronic diseases, the predicted BA was higher than the CA when compared to the age difference in healthy participants by AgeDiff of 3.16 years in CHD (95% CI, 2.67-3.62: p-value<0.001), 3.85 years in CKD (95% CI, 3.43-4.35: p-value <0.001), 4.51 years in CVD (95% CI, 3.77-5.23: p-value<0.001), 3.94 years in diabetes (95% CI, 3.58-4.43: p-value<0.001), 4.06 years in hypertension (95% CI, 3.74-4.33: p-value<0.001), and 4.94 years in stroke (95% CI, 4.13-5.48: p-value<0.001). Interestingly, a AgeDiff of 5.43 years was observed in smokers (95% CI, 4.56-6.13: p-value<0.001), AgeDiff of 3.62 years in drinkers (95% CI, 3.45-4.16: p-value<0.001), and a AgeDiff of 4.36 years in obese participants (BMI>27, 95% CI, 3.71-4.82: p-value<0.001).

### Example 4—Prediction of Chronic Diseases Risks Using AgeDiff

The difference between BA and CA was categorized into 4 equal quartiles in an attempt to stratify the analyses on the basis of the BA difference. The hazard ratio (HR) of developing each of the chronic diseases in each of these quartiles was evaluated. The results of the analyses are shown in Table 2. Overall, changes in the AgeDiff were associated with the development of any types of the six chronic diseases in the internal cohort (HR=1.5, 95% CI=1.70-2.11, P=0.015) and external cohort (HR=1.4, 95% CI=1.10-1.63, P=0.031). For the individual chronic diseases evaluated, changes in BA were associated with an increased HR for developing each of the diseases analysed in the internal cohort (hypertension, CHD, diabetes, CVD, stroke and CKD) and external cohort (hypertension, CHD, CVD, diabetes and stroke). Within the different quartiles, there was an overall trend for increasing HR for developing each of the chronic diseases with successive quartiles. In both cohorts, quartile 4 was significantly associated with higher HR for developing chronic diseases, while there were no significant associations in quartiles 1 and 2. In the internal cohort, patients in quartile 3 were significantly associated with higher HR for diabetes and stroke, while external participants in quartile 3 were significantly associated with CVD. The association between BA difference and HR for developing these common chronic diseases remained statistically significant even following the removal of participants who were diagnosed with these diseases within one year (Table 4).

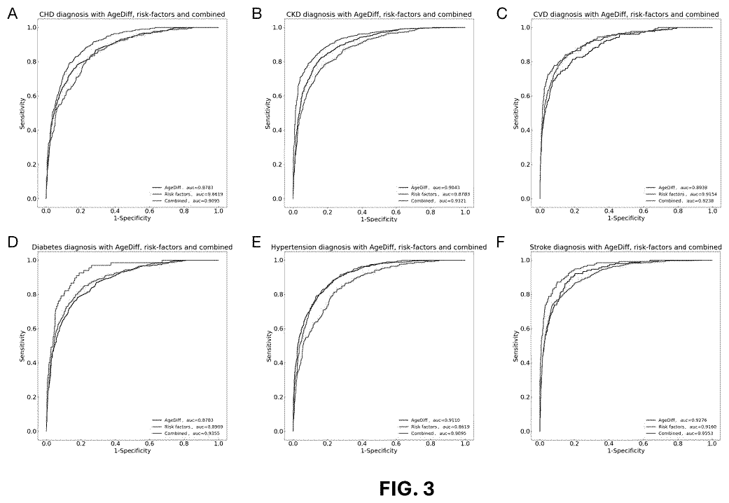

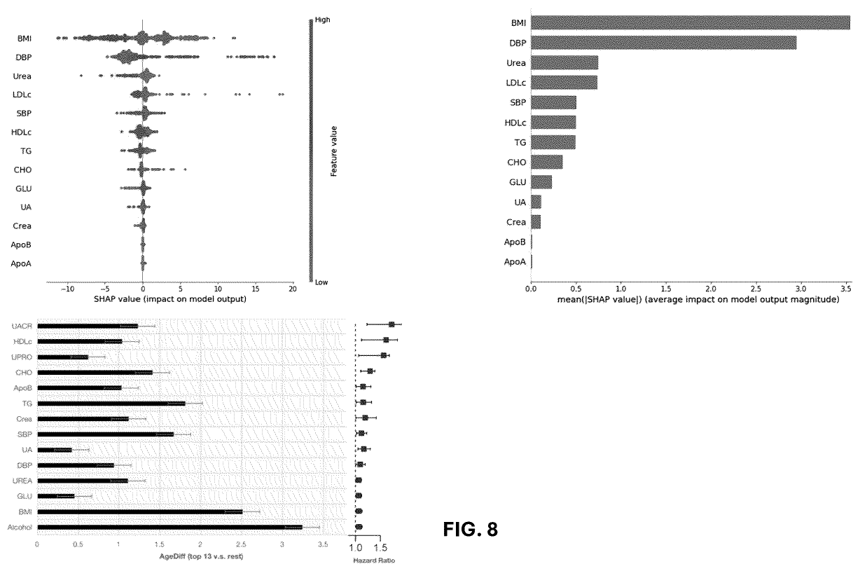

The utility of the multi-modal image fusion model on AgeDiff was then evaluated to predict the 5-year risks of developing CHD, CVD, CKD, stroke, hypertension and diabetes, and compared these predictions to standard approaches using established risk factors. Among these risk factors, the body-mass index and diastolic blood pressure was found to have the largest impact on predicted BA, using SHAP analyses (FIG. 8). The receiver operator characteristic (ROC) curves for prediction of chronic disease development are shown in FIG. 3. The predictive value of the AgeDiff for chronic diseases, evaluated using area under the curve (AUC) measurements, was found to be consistently higher, relative to predictions using risk factors. Importantly, the combination of BA difference with risk factors improved the AUC, indicating that BA prediction can be used in conjunction with existing risk factors to identify individuals at risk of developing chronic disease.

### Example 5—Incidence Prediction of Chronic Diseases Using AgeDiff

The results so far demonstrate that BA difference can be used to predict the risk of developing chronic diseases. Next, the BA prediction model was evaluated for its use in predicting the disease onset. The performance of incidence prediction for different chronic diseases using BA difference under the Cox proportional hazards (CPH) model is summarized in Table 3, showing the performance of progression prediction model to six common chronic systematic diseases event based on the risk-factor-only model, and the combined model (including multi-modal images and risk-factors) on the internal and external test sets. Concordance index (C-index) for right-censored data and 95% CI measure the model performance by comparing the progression information (disease labels and progression days) with predicted risk scores. A larger Cndex correlates with better progression prediction performance. CI, confidence interval.

Similar to the observations above, combination of the BA difference and the risk-factor based model provided an improved C-index for the incidence detection of chronic diseases. When testing on another independent external cohort, similar results were observed. The above results show that BA, as an important biomarker, could be used to assist existing factors for disease prognosis.

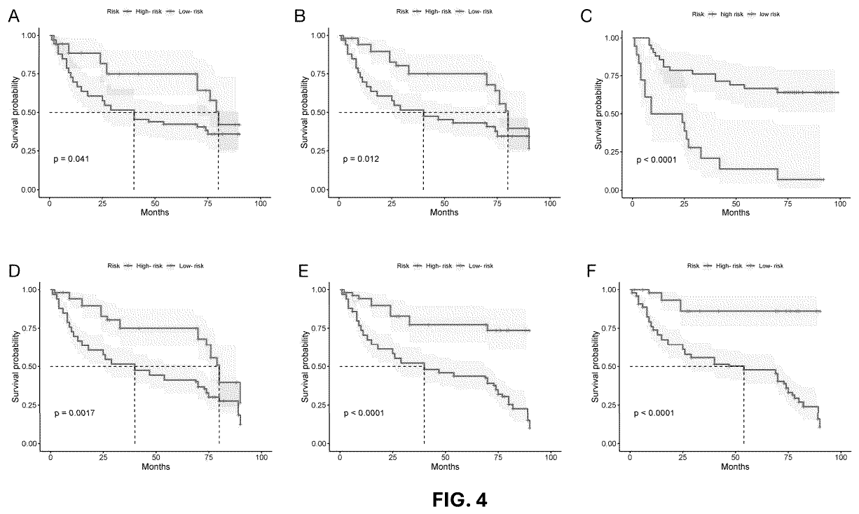

The Kaplan-Meier method was used to stratify healthy individuals at the baseline into two risk groups (low or high risk) for developing chronic diseases. The incidence of the different diseases stratified by risk groups of the BA difference model is shown in FIG. 4. For the Kaplan-Meier curves and log-rank tests, thresholds for the high-risk and low-risk groups were based on the upper and lower quartiles of the predicted risk scores from the combined models in the training cohort. The approach was then tested on the test cohort and found statistically significant separations of the low-risk and high-risk groups. The data therefore indicate that the multi-modal image fusion AI model was able to identify at-risk patients for chronic diseases and predict chronic disease incidence.

### Example 6—Relations Between AgeDiff and Other Risk Factors

According to previous studies [16], [17], the six chronic diseases included in this study have been associated with various risk factors, among which 61 covariates were collected. Univariate and multivariate survival analyses were conducted using Cox proportional hazards methods (likelihood ratio), including AgeDiff and other prognostic factors, in addition to the scores generated from the six chronic diseases. As Table 5 shows, under both univariate and multivariate analysis, AgeDiff is proved to be a significant factor for developing chronic diseases. The relations between AgeDiff and other risk factors were further investigated, including the most relevant risk factors related to AgeDiff. To this end, lightgbm [18] was built to use a gradient boosting framework that uses tree based learning algorithms for mapping 61 factors to AgeDiff. FIG. 8 shows top 13 variants in terms of attributable AgeDiff using shapley additive explanations (SHAP) method [19] (left and middle). The top 13 variants were also illustated in terms of attributable AgeDiff and HRs for any diseases (right). These results provide explainable contributors to AgeDiff and chronic diseases.

### Example 7—Relations Between AgeDiff and Other Risk Factors

The aging process is inevitable and is a risk factor for chronic diseases. The biological age (BA) of each individual contains structural and functional determinants of aging, and its difference (AgeDiff) from the chronological age (CA) can be used as a biomarker for accelerated aging caused by underlying pathologies. Described herein in this example is a multimodal Transformer-based architecture which can estimate BA based on facial, fundus, and tongue images. The results demonstrated that the model can accurately estimate BA of healthy individuals, significant deviations of AgeDiff are present in individuals with chronic diseases, and AgeDiff can be used to accurately detect systematic diseases and identify progression risks. The results highlight an approach to use easily and readily acquired patient data to identify chronic diseases.

### Image Datasets and Patient Characteristics

The 3D facial, tongue and retinal images were collected from the study cohorts of the China Bioage Investigation Consortium (CBIC), which consists of the following participants: the northern China cohort which was used for the model training and the southern China cohort, which is used for an independent validation. The northern China cohort is from the China suboptimal health cohort study (COACS) in Tangshan City, Heibei Province, China. The southern China cohort is from the Nanfang Hospital in Guangzhou, Guangdong Province, Zhuhai People's Hospital/the first affiliated Hospital of MUST. Institutional Review Board (IRB)/Ethics Committee approvals were obtained in all locations and all participating subjects signed an informed consent form.

The COACS is a community-based, prospective study; to investigate how suboptimal health status contributes to the incidence of non-communicable chronic diseases in Chinese adults [33]. This COACS study is a cross-sectional survey. The participants were recruited from Tangshan city, which is a large, modern industrial city adjacent to two megacities: Beijing and Tianjin. All participants underwent clinical, laboratory and environmental exposure measurements aimed at identifying clinical, biological, environmental, and genetic factors associated with suboptimal health. This cohort was used for the study because it has the balance of healthy subjects and those with metabolic diseases, medical records were relatively complete, and previous electronic medical records were available for assessment if needed. The southern China cohort is also a community-based, annual health-check prospective study with a similar study design.

The northern China developmental cohort and the southern China validation external cohort consisted of patients with demographic information and clinical parameters from their electronic medical records. If they consented to this study, they were subjected to 3D face, tongue and retina scanning, fasting blood draws, and the use of medical record data. 3D facial images were captured using 3dMDface camera systems (www.3dmd.com) with the study beginning in their annual visit in 2018-2022. Applying standard facial and retina image acquisition protocols, participants were asked to close their mouths and hold their faces with a neutral expression for the capture of the digital facial stereophotogrammetry. 3D images in wavefront.obj file format with point clouds and corresponding texture images were used for further analysis. For each consenting subject, demographic, routine physical examination, and clinical laboratory were obtained. Demographic and clinical data for all the study participants are summarized in Table 1.

### Data Pre-Processing

The multi-modal-fusion architecture received three inputs, the integrated tongue, retinal fundus and facial images. The size of each image was resized to 256×256. Tongue and facial images included learnable parameters that were optimized along with the multi-modal-fusion architecture.

Tongue images in this study were captured using standard settings on an iphone X. Samples which were corrupt, vague, or those with strong illumination were excluded from the analysis. Non-tongue elements, such as the face, teeth, lips and neck were removed using a pre-processing segmentation step. This involved coarse segmentation and fine segmentation to obtain pixel-level tongue contour, which is superior to rectangular ROI detection approaches. Rectangular ROI was produced in the coarse step, which formed the input for the fine segmentation. The de-correlation stretch algorithm [28] used was equipped with OSTU method to attain an edge map. The tongue contour obtained from the improved maximal similarity-based region merging (MSRM) method [35] was then combined with the edge map to generate a weight map of the equal size to the original tongue image. Finally, the edge-based method fast marching [36] was implemented on the weight map to compute the final tongue contour. Once a precise tongue contour was obtained, it was converted into three spaces using three learnable modules (ColorNet, TextureNet and Geometry Net), and leveraged their integrated image as the input of the multi-modal-fusion architecture. ColorNet consists of three multi-layer perceptrons (MLPs) which take as input the conversion output from the original RGB contour using standard RGB-CIE mapping. TextureNet consists of three MLPs which take as input the RGB channels. Geometry Net consists of a combination of two sub-networks, where the first is a three MLPs that receive the gray version of the contour and the second is a linear embedding that takes as input the key landmark points [37].

The retinal fundus images were captured using standard fundus cameras, including Topcon TRC-NW6 (Topcon), Zeiss Visucam 224 (Carl Zeiss Meditec AG), Canon CR6-45NM (Canon) and KOWA Nonmyd α-DIII (Kowa). All fundus images were de-identified. For screening and grading retinal fundus images, a hierarchical two-tier grading process was performed by ten phase I and five phase II graders. Phase I graders consisted of individuals trained by ophthalmologists and evaluated to attain at least 95% accuracy determined by a quiz consisting of 1,000 fundus images of various retinal diseases. Phase II graders consisted of ophthalmologists who individually reviewed every image classified by phase I graders. To check consistency among phase II graders, 20% of images were randomly selected and reviewed by three senior retinal specialists. The second tier of five ophthalmologists independently read and verified the true labels for each image. To account for disagreement, the evaluation test set was also checked by expert consensus.

The 3D facial stereophotogrammetry images were captured with standard acquisition protocols, where participants were asked to close their mouths and hold their faces with a neutral expression. Each 3D facial image included a 3D mesh and a corresponding texture image, extracted for each point and constructed into an integrated facial image as the input of multi-modal-fusion architecture. The texture features were expressed with the color of each point in a 3D facial image mapped through captured 2D texture images and texture coordinates to describe the photometric and color attributes of the face. Geometry features include global geometry features and local geometry features. Global features included the sizes of the whole mesh and feature map of each component with three channels comprising the 3D coordinates of each point. Local features included shape depressions and prominences that were quantified by normal vectors and surface curvatures at each point in the mesh. Gaussian curvature and mean curvature of curvature for each point was calculated. Finally, global and local geometry maps as well as texture maps were integrated to generate a facial image.

### Multi-Modal-Fusion Architecture Settings

The number of multi-modal Transformer encoders K was set to 3. The numbers of Swin-Transformer [38] encoders for each modality were set to M=4, N=4 and K=5. The number of Swin-Transformer encoders of cross-attention modules in one multi-modal Transformer encoder was set to L=3. The expanding ratio of feed-forward network in the Swin-Transformer encoder was set to 4. The number of headers were the same and set to 3 for three branches. Each of the two hidden layers in MLP had 128 nodes and was applied with the rectified linear unit (ReLU) activation function. The Mean-Square Error (MSE) loss was used as an objective function for the regression task of numerical value prediction between BA and CA. Other settings follow the default of Swin-Transformer V2.

The multi-modal fusion architecture training details were as follows. Transformations of random horizontal flip and rotations limited to +20 degrees were added to each batch during training as data augmentation to enable an improved and generalized network learning. AdamW optimizer [39] was used and cosine learning rate decay policy with an initial learning rate of 0.001. 8 Telsa-A100 GPUs were used and the model was trained for 350 epochs using Pytorch [40] library. The batch size was set to 256. 5 epochs for learning rate warm-up were used [41]. Mixup [42] and random augmentation [43] techniques were used to boost the performance. R kesults on the test set were reported using the optimal hyper-parameters of the architecture selected in a grid search manner on the validation set.

### Definition of AgeDiff and Criteria for Disease Diagnosis

The age gap was defined as the difference between the predicted BA age using multi-modal-fusion method and CA, where a positive age gap indicates a biological aging faster than the patient's CA, while a negative biological age gap suggests that the biological ages slower. The following criteria were used to define systemic diseases. CKD was defined as an eGFR of more than 60 ml min⁻¹ per 1.73 m² with albuminuria or less than 60 ml min⁻¹ per 1.73 m², confirmed in at least two visits separated by three months. Healthy controls were defined as eGFR above 60 ml min⁻¹ per 1.73 m² without albuminuria, determined using a negative urine dip-stick test. Diabetes was defined by a fasting blood glucose ≥7.0 mmol l⁻¹ at least two times, an HbA1c value of 6.5% or more and/or a history of drug treatment for diabetes. Hypertension was defined as a persistent increase in blood pressure above 130/80 or 140/90 mm Hg. Smoking as a risk factor was defined as participants smoke 5 cigarettes per day averagely.

### Prediction of the Incidence Development of Systematic Diseases Using Longitudinal Cohorts

For the incidence analysis of each disease, the index data was denoted as the time without disease (at baseline). The development of each disease was evaluated as an incidence data (or end-point) within the yearly clinical follow-up. The CPH models were trained on the training and tuning set using variables based on the metadata and multi-modal-image-based risk score. The metadata-based model comprised sex, BMI, height, weight, smoking, SBP, DBP, eGFR and blood glucose. The multi-modal-image-based risk core is the predicted z-score (standard score) of the first visit generated from the detection model of each disease and used to predict progression risks of patients in combination with metadata. According to the risk scores of the first visit from the CPH model for the detection of each disease, the patients are triaged into three groups: low; medium and high risk according to the upper and lower quartiles of predicted risk scores in the tuning set, respectively. Table 2 shows the distribution of the risk scores and the related thresholds (the upper and lower quartiles) across datasets. The risk scores were also treated as categorical variables according to quartiles during the incidence analysis on validation sets. Kaplan-Meier curves were constructed for the risk groups, and the significance of differences between group curves was computed using the log-rank test. Time-dependent ROC curves were used to quantify model performance on validation sets at the time of interest. ROC curves were constructed at a landmark time from predicted risk scores of relative patients made using the model. The univariable and multivariable CPH models were fitted. Two multivariable CPH models were developed, a combined metadata and fundus model and a metadata-only model serving as a baseline model. Statistical significance of HRs and adjusted HRs of CPH models were evaluated using the likelihood ratio test.

### Interpretation of AI Predictions

The Grad-CAM++ method was used to produce visual explanations. Grad-CAM++ provides pixel-wise weighting of the gradients of the output with respect to a particular spatial position in any feature map of a DL-based system. In a single backward pass on the computational graph, a measure of importance of each pixel in a feature map towards the overall decision of the system was shown. In the scenario, the gradients of age difference between BA and CA were back-propagated through three MLP headers, multi-modal Transformer encoders and linear projections to three input modalities. The saliency maps generated by Grad-CAM++ indicate the effect of each pixel on the model predictions. Gaussian filtering was applied to saliency maps for smoothness on three input modalities images. FIG. 5 shows an example of Grad-CAM++ results on three-modality inputs of one participant on internal training set in the training process. The saliency maps in the training process gradually provide visual clues on different regions of face, fundus and tongue.

### Statistical Analysis

To evaluate the performance of regression models for continuous values prediction (age) in this study, MAE, R² and PCC were calculated. The Bland-Altman plot was applied to display the difference between CA and the predicted value of BA against the average of the two. With 95% limits of agreement and ICC, the agreement of the predicted BA and CA was evaluated. The ratio between the variance of the model outputs and the variance of real-world data was calculated using the tuning set to calibrate outputs. Sensitivity and specificity were determined by the selected thresholds on the validation set. The models' performance on binary classification predictions was evaluated by ROC curves of sensitivity versus 1-specificity. The AUC of ROC curves were reported with 95% CI. The 95% CI of AUCs were estimated with the non-parametric bootstrap method (1,000 random resampling with replacement). The detection of each disease using BA were evaluated with binary classification models. The incidence rate for the whole cohort was calculated and for each risk group as the number of events per 1,000 person-years at risk. The Byar Poisson approximation method was used to calculate 95% CI of incidence [46]. Then Kaplan-Meier estimators were constructed for different risk groups, and the significance of differences between groups was tested by log-rank tests. CPH models were tested using the likelihood ratio test. The time-dependent AUC was used at four years and five years to measure model performance. The Kaplan-Meier curve and the time-dependent ROC-AUC were calculated using the Python packages of lifelines (version 0.27.4) and scikit-survival (version 0.19.0).

TABLE 1
basic characteristics of the participants in the internal data set and the external data set.

| | | | | |
|---|---|---|---|---|
| Cohorts | Normal | Any Disease | CHD | CKD |

Northern China cohort

| Participants | 11223 | | 2136 | | 321 | | 935 | |
|---|---|---|---|---|---|---|---|---|
| Image | 55948 | | 10846 | | 1622 | | 4448 | |
| Face | 21332 | | 7406 | | 610 | | 1706 | |
| Fundus | 21140 | | 7340 | | 606 | | 1684 | |
| Tongue | 13456 | | 4174 | | 406 | | 1058 | |
| Female (%) | 5846 | (52%) | 983 | (46%) | 142 | (44%) | 452 | (48%) |
| Age (yr) | 53.8 ± 11.3 | | 56.7 ± 10.5 | | 55.6 ± 10.9 | | 57.2 ± 11.2 | |
| BMI (kg/m²) | 24.7 ± 2.3 | | 25.0 ± 2.4 | | 24.9 ± 2.2 | | 24.8 ± 2.4 | |
| Smoking (%) | 2531 | (23%) | 1329 | (62%) | 171 | (53%) | 379 | (41%) |
| Drinking (%) | 3716 | (33%) | 1405 | (66%) | 142 | (44%) | 514 | (55%) |

| | | | | |
|---|---|---|---|---|
| cGFR (ml/min per 1.73 m²) | 97.3 ± 22.5 | 101.5 ± 23.8 | 98.2 ± 22.9 | 103.2 ± 24.5 |
| Blood glucose (mmol/l) | 6.6 ± 2.3 | 7.1 ± 2.5 | 6.9 ± 2.8 | 7.0 ± 2.6 |

Southern China cohort

| Participants | 2840 | | 630 | | 43 | | — |
|---|---|---|---|---|---|---|---|
| Image | 8600 | | 2867 | | 183 | | — |
| Face | 1922 | | 910 | | 55 | | — |
| Fundus | 4440 | | 1216 | | 86 | | — |
| Tongue | 2238 | | 905 | | 52 | | — |
| Female (%) | 844 | (29.7%) | 98 | (26.7%) | 11 | (34.8%) | — |
| Age (yr) | 49.8 ± 7.3 | | 56.2 ± 9.8 | | 55.4 ± 9.8 | | — |
| BMI (kg/m²) | 24.2 ± 3.6 | | 24.9 ± 3.3 | | 24.6 ± 3.2 | | — |
| Smoking (%) | 762 | (26.4%) | 168 | (26.8%) | 7 | (16.3%) | — |
| Drinking (%) | 119 | (42.0%) | 264 | (42.1%) | 142 | (44.0%) | — |

| Blood glucose (mmol/l) | 5.6 ± 1.7 | 6.2 ± 2.1 | 5.7 ± 1.7 | — |
|---|---|---|---|---|
| Cohorts | CVD | Diabetes | Hypertension | Stroke |
| | Northern China cohort | | | |
| Participants | 354 | 323 | 1686 | 57 |
| Image | 1906 | 2296 | 8480 | 280 |
| Face | 702 | 11004 | 3280 | 104 |
| Fundus | 692 | 998 | 3256 | 104 |
| Tongue | 158 | 694 | 1944 | 72 |

| | | | | | | | | |
|---|---|---|---|---|---|---|---|---|
| Female (%) | 158 | (44%) | 249 | (47%) | 823 | (49%) | 25 | (44%) |

| | | | | |
|---|---|---|---|---|
| Age (yr) | 57.3 ± 10.8 | 56.6 ± 11.4 | 55.1 ± 10.8 | 57.4 ± 11.0 |
| BMI (kg/m²) | 25.1 ± 2.2 | 25.0 ± 2.3 | 25.1 ± 2.3 | 25.2 ± 2.2 |

| | | | | | | | | |
|---|---|---|---|---|---|---|---|---|
| Smoking (%) | 140 | (40%) | 325 | (62%) | 896 | (53%) | 3 | (5%) |
| Drinking (%) | 153 | (43%) | 318 | (61%) | 1045 | (62%) | 9 | (16%) |

| cGFR (ml/min per 1.73 m²) | 99.3 ± 22.0 | 100.6 ± 20.7 | 99.3 ± 23.1 | 98.3 ± 20.5 |
|---|---|---|---|---|
| Blood glucose (mmol/l) | 7.2 ± 2.3 | 7.1 ± 2.8 | 7.0 ± 2.9 | 6.9 ± 2.6 |
| | Southern China cohort | | | |
| Participants | 36 | 124 | 510 | 36 |
| Image | 155 | 503 | 2038 | 142 |
| Face | 40 | 156 | 614 | 45 |
| Fundus | 72 | 204 | 793 | 61 |
| Tongue | 43 | 143 | 631 | 36 |

| | | | | | | | | |
|---|---|---|---|---|---|---|---|---|
| Female (%) | 7 | (19.4%) | 30 | (24.2%) | 134 | (26.3%) | 7 | (19.4%) |

| | | | | |
|---|---|---|---|---|
| Age (yr) | 57.3 ± 10.8 | 57.2 ± 9.9 | 55.1 ± 10.8 | 57.4 ± 11.0 |
| BMI (kg/m²) | 24.8 ± 3.4 | 24.0 ± 3.7 | 25.4 ± 3.6 | 24.8 ± 3.4 |

| | | | | | | | | |
|---|---|---|---|---|---|---|---|---|
| Smoking (%) | 12 | (33.3%) | 34 | (27.4%) | 125 | (24.5%) | 12 | (33.3%) |
| Drinking (%) | 13 | (36.1%) | 38 | (30.6%) | 200 | (41.0%) | 13 | (36.1%) |

| | | | | |
|---|---|---|---|---|
| Blood glucose (mmol/l) | 6.1 ± 2.0 | 7.6 ± 2.8 | 5.7 ± 1.4 | 6.1 ± 2.0 |

TABLE 2
The association between the AgeDiff with the incident of six common chronic systematic diseases. The first quartile (Q1) is
defined as the set of data between the smallest value and the 25th retinal age gap. The second quartile (Q2) is the set of
data between the 25th and median value. The third quartile (Q3) is set of data between the median value and the 75th retinal
age gap. The fourth quartile (Q4) is defined as the set of data between the 75th and the maximum of the retinal age gap.

| Cohorts | | | |
|---|---|---|---|
| Any Disease | CHD | CKD | CVD |

Internal test set AgeDiff

| All participants | | HR (95% CI) | P-Value | HR (95% CI) | P-Value | HR (95% CI) | P-Value | HR (96% CI) | P-Value |
|---|---|---|---|---|---|---|---|---|---|
| Mean (SD) | 2.32 (4.56) | 1.5 (1.31-2.11) | 0.015 | 1.9 (1.70-2.21) | 0.031 | 1.1 (1.02-1.32) | 0.018 | 1.4 (1.12-1.69) | 0.023 |
| Quartile 1 | −7.23 (3.05) | 1 | — | 1 | — | 1 | — | 1 | — |
| | | [Reference] | | [Reference] | | [Reference] | | [Reference] | |
| Quartile 2 | −2.59 (1.31) | 1.34 (1.13-1.51) | 0.116 | 1.72 (1.43-1.91) | 0.108 | 1.32 (1.09-1.46) | 0.043 | 1.23 (1.09-1.17) | 0.192 |
| Quartile 3 | 4.18 (1.78) | 2.15 (1.74-2.53) | 0.024 | 1.76 (1.24-2.23) | 0.048 | 2.57 (1.91-3.62) | 0.012 | 2.96 (1.94-3.55) | 0.041 |
| Quartile 4 | 8.25 (2.70) | 5.72 (4.59-6.11) | 0.007 | 5.04 (4.29-6.42) | 0.022 | 5.25 (4.41-6.06) | 0.005 | 5.16 (4.34-5.74) | 0.010 |

External test set AgeDiff

| All participants | | HR (95% CI) | P-Value | HR (95% CI) | P-Value | — | — | HR (95% CI) | P-Value |
|---|---|---|---|---|---|---|---|---|---|
| Mean (SD) | 2.07 (4.13) | 1.4 (1.10-1.62) | 0.031 | 1.6 (1.18-1.73) | 0.071 | — | — | 1.6 (1.31-1.77) | 0.013 |
| Quartile 1 | −8.12 (3.43) | 1 | — | 1 | — | — | — | 1 | — |
| | | [Reference] | | [Reference] | | | | [Reference] | |
| Quartile 2 | −4.29 (1.85) | 1.55 (1.22-1.74) | 0.046 | 1.72 (1.43-1.91) | 0.112 | — | — | 1.43 (1.12-1.63) | 0.132 |
| Quartile 3 | 2.13 (1.72) | 1.87 (1.44-2.15) | 0.015 | 3.06 (2.39-3.63) | 0.029 | — | — | 2.43 (1.74-3.10) | 0.021 |
| Quartile 4 | 6.35 (2.32) | 4.67 (3.34-6.52) | 0.002 | 5.53 (3.81-6.79) | 0.004 | — | — | 4.16 (3.64-5.29) | 0.009 |

| | | Cohorts | | | | | |
|---|---|---|---|---|---|---|---|
| | | Diabetes | | Hypertension | | Stroke | |
| Internal test set AgeDiff | | | | | | | |
| All participants | | HR (95% CI) | P-Value | HR (95% CI) | P-Value | HR (95% CI) | P-Value |
| Mean (SD) | 2.32 (4.56) | 1.5 (1.26-1.77) | 0.042 | 2.0 (1.74-2.14) | 0.028 | 1.3 (1.09-1.44) | 0.041 |
| Quartile 1 | −7.23 (3.05) | 1 | — | 1 | — | 1 | — |
| | | [Reference] | | [Reference] | | [Reference] | |
| Quartile 2 | −2.59 (1.31) | 1.33 (1.13-1.71) | 0.113 | 1.45 (1.15-1.72) | 0.071 | 1.62 (1.23-1.81) | 0.194 |
| Quartile 3 | 4.18 (1.78) | 2.36 (1.42-3.31) | 0.035 | 2.61 (1.94-3.60) | 0.043 | 2.35 (1.65-3.31) | 0.038 |
| Quartile 4 | 8.25 (2.70) | 5.61 (4.52-6.35) | 0.026 | 5.78 (4.71-7.21) | 0.021 | 4.67 (4.10-5.53) | 0.018 |
| External test set AgeDiff | | | | | | | |
| All participants | | HR (95% CI) | P-Value | HR (95% CI) | P-Value | HR (95% CI) | P-Value |
| Mean (SD) | 2.07 (4.13) | 1.3 (1.12-1.74) | 0.038 | 1.7 (1.84-2.05) | 0.037 | 1.1 (1.04-1.32) | 0.025 |
| Quartile 1 | −8.12 (3.43) | 1 | — | 1 | — | 1 | — |
| | | [Reference] | | [Reference] | | [Reference] | |
| Quartile 2 | −4.29 (1.85) | 1.33 (1.13-1.71) | 0.103 | 1.45 (1.15-1.72) | 0.043 | 1.54 (1.28-1.79) | 0.033 |
| Quartile 3 | 2.13 (1.72) | 2.28 (1.73-3.04) | 0.025 | 3.41 (2.13-3.78) | 0.013 | 2.65 (1.85-3.21) | 0.011 |
| Quartile 4 | 6.35 (2.32) | 4.61 (4.05-6.24) | 0.016 | 5.68 (4.71-7.21) | 0.008 | 5.67 (4.10-6.56) | 0.005 |

TABLE 3
Performance of progression prediction model to six common chronic systematic diseases
event based on the risk- factor- only model, and the combined model (including
multi- modal images and risk- factors) on the internal and external test sets.

| | Progression prediction models | C-index on internal test set | C-index on external test set |
|---|---|---|---|
| CHD | Risk-factor-based model | 0.775 (95% CI: 0.719-0.850) | 0.813 (95% CI: 0.726-0.853) |
| | BA-based model | 0.825 (95% CI: 0.726-0.894) | 0.848 (95% CI: 0.751-0.896) |
| | Combined model | 0.853 (95% CI: 0.812-0.913) | 0.872 (95% CI: 0.830-0.925) |
| CKD | Risk-factor-based model | 0.828 (95% CI: 0.753-0.916) | — |
| | BA-based model | 0.813 (95% CI: 0.734-0.904) | — |
| | Combined model | 0.865 (95% CI: 0.768-0.935) | — |
| CVD | Risk-factor-based model | 0.806 (95% CI: 0.731-0.901) | 0.803 (95% CI: 0.753-0.861) |
| | BA-based model | 0.819 (95% CI: 0.758-0.896) | 0.841 (95% CI: 0.788-0.899) |
| | Combined model | 0.856 (95% CI: 0.788-0.924) | 0.857 (95% CI: 0.801-0.905) |
| Diabetes | Risk-factor-based model | 0.868 (95% CI: 0.761-0.915) | 0.803 (95% CI: 0.751-0.882) |
| | BA-based model | 0.867 (95% CI: 0.772-0.927) | 0.857 (95% CI: 0.781-0.905) |
| | Combined model | 0.903 (95% CI: 0.824-0.942) | 0.872 (95% CI: 0.814-0.933) |
| Hypertension | Risk-factor-based model | 0.813 (95% CI: 0.712-0.890) | 0.803 (95% CI: 0.743-0.866) |
| | BA-based model | 0.826 (95% CI: 0.735-0.912) | 0.826 (95% CI: 0.778-0.894) |
| | Combined model | 0.874 (95% CI: 0.788-0.939) | 0.854 (95% CI: 0.792-0.915) |
| Stroke | Risk-factor-based model | 0.872 (95% CI: 0.773-0.920) | 0.810 (95% CI: 0.753-0.864) |
| | BA-based model | 0.861 (95% CI: 0.756-0.917) | 0.834 (95% CI: 0.796-0.894) |
| | Combined model | 0.895 (95% CI: 0.842-0.935) | 0.876 (95% CI: 0.821-0.921) |

TABLE 4
Predicted incidence rates of six common chronic systematic diseases (per 1,000 person-years) for the
in-ternal longitudinal test set and for the external longitudinal test set, stratified by risk level.

| | | | | | Univariate Analysis | | Multivariate Analysis | |
|---|---|---|---|---|---|---|---|---|
| Disease | Subset | Participants | Events | Incident Rate (95% CI) | HR (95% CI) | P value | HR (95% CI) | P value |

Prognostic analysis on internal longitudinal test set

| | | | | | | | | |
|---|---|---|---|---|---|---|---|---|
| CHD | | | | | | | | |
| | Low risk | 1029 | 31 | 3.0 (0.6, 9.5) | Reference | NA | Reference | NA |
| | High risk | 1063 | 94 | 8.6 (3.8, 17.8) | 5.7 (2.4, 8.0) | <0.001 | 2.2 (0.9, 5.3) | <0.001 |
| CKD | | | | | | | | |
| | Low risk | 1854 | 102 | 5.5 (1.3, 9.6) | Reference | NA | Reference | NA |
| | High risk | 1771 | 280 | 15.8 (4.9, 23.4) | 9.2 (3.3, 14.5) | <0.001 | 6.4 (3.8, 9.6) | <0.001 |
| CVD | | | | | | | | |
| | Low risk | 1317 | 25 | 1.9 (0.1, 4.1) | Reference | NA | Reference | NA |
| | High risk | 1392 | 77 | 5.5 (3.8, 8.5) | 3.1 (0.7, 5.6) | <0.001 | 1.7 (0.3, 4.2) | <0.001 |
| Diabetes | | | | | | | | |
| | Low risk | 1648 | 55 | 3.3 (0.5, 6.7) | Reference | NA | Reference | NA |
| | High risk | 1715 | 110 | 6.4 (5.8, 11.5) | 2.6 (1.1, 4.6) | <0.001 | 2.1 (1.3, 3.2) | <0.001 |
| Hypertension | | | | | | | | |
| | Low risk | 2683 | 157 | 6.0 (2.1, 9.4) | Reference | NA | Reference | NA |
| | High risk | 3297 | 316 | 9.6 (5.8, 15.5) | 5.3 (2.4, 8.0) | <0.001 | 3.7 (1.6, 5.2) | <0.001 |
| Stroke | | | | | | | | |
| | Low risk | 1492 | 11 | 0.7 (0.0, 2.7) | Reference | NA | Reference | NA |
| | High risk | 1384 | 5 | 0.3 (0.0, 2.4) | 2.3 (1.8, 2.8) | <0.001 | 1.9 (1.4, 2.4) | <0.001 |

Prognostic analysis on external longitudinal test set

| | | | | | | | | |
|---|---|---|---|---|---|---|---|---|
| CHD | | | | | | | | |
| | Low risk | 169 | 8 | 2.3 (1.4, 3.5) | Reference | NA | Reference | NA |
| | High risk | 177 | 13 | 5.3 (1.9, 4.7) | 4.7 (2.1, 7.5) | <0.001 | 3.2 (1.9, 5.1) | <0.001 |
| CVD | | | | | | | | |
| | Low risk | 125 | 5 | 1.7 (1.1, 2.7) | Reference | NA | Reference | NA |
| | High risk | 332 | 16 | 4.5 (3.8, 8.5) | 3.3 (1.7, 5.2) | <0.001 | 2.7 (0.9, 4.8) | <0.001 |
| Diabetes | | | | | | | | |
| | Low risk | 204 | 32 | 4.5 (1.5, 7.9) | Reference | NA | Reference | NA |
| | High risk | 425 | 45 | 7.4 (3.8, 12.2) | 4.3 (1.4, 5.2) | <0.001 | 4.1 (1.2, 4.7) | <0.001 |
| Hypertension | | | | | | | | |
| | Low risk | 191 | 72 | 6.0 (4.1, 8.2) | Reference | NA | Reference | NA |
| | High risk | 367 | 151 | 11.6 (5.1, 16.2) | 6.4 (3.3, 8.9) | <0.001 | 4.3 (2.6, 5.9) | <0.001 |
| Stroke | | | | | | | | |
| | Low risk | 152 | 4 | 1.1 (0.1, 2.9) | Reference | NA | Reference | NA |
| | High risk | 324 | 8 | 2.4 (0.1, 2.5) | 2.9 (1.4, 4.1) | <0.001 | 2.1 (1.5, 2.7) | <0.001 |

TABLE 5
Univariate and multivariate survival analyses of six common chronic systematic diseases
conducted using Cox proportional hazards (CPH) methods (likelihood ratio test).

| | | Univariate analysis | | Multivariate analysis | | | Univariate analysis | | Multivariate analysis | |
|---|---|---|---|---|---|---|---|---|---|---|
| Covariates | Disease | HR (95% CI) | P-Value | HR (95% CI) | P-Value | Disease | HR (95% CI) | P-Value | HR (95% CI) | P-Value |
| | CHD | | | | | Diabetes | | | | |
| Sex | | 0.94 (0.71-0.90) | <0.001 | 0.91 (0.73-1.07) | <0.001 | | 0.65 (0.56-0.76) | <0.001 | 1.01 (0.82-1.24) | <0.001 |
| BMI | | 1.24 (1.06-1.31) | <0.001 | 1.14 (1.04-1.21) | <0.001 | | 1.16 (1.14-1.18) | <0.001 | 1.08 (1.04-1.11) | <0.001 |
| Height | | 0.91 (0.74-0.98) | <0.001 | 0.88 (0.70-0.99) | <0.001 | | 1.00 (0.99-1.01) | 0.077 | 0.99 (0.98-1.01) | 0.053 |
| Weight | | 1.44 (1.13-1.51) | 0.014 | 1.34 (1.01-1.42) | 0.033 | | 1.03 (1.03-1.04) | <0.001 | 1.02 (1.00-1.03) | <0.001 |
| Smoking | | 1.77 (1.45-2.62) | 0.017 | 1.52 (1.12-1.93) | 0.028 | | 1.76 (1.36-2.28) | <0.001 | 1.68 (1.33-2.12) | <0.001 |
| SBP | | 1.13 (1.01-1.19) | 0.024 | 1.03 (1.00-1.08) | 0.015 | | 2.37 (1.64-3.11) | <0.001 | 2.21 (1.55-2.93) | <0.001 |
| DBP | | 1.17 (1.04-1.42) | <0.001 | 1.06 (1.01-1.15) | <0.001 | | 2.01 (1.34-2.11) | <0.001 | 1.93 (1.36-2.21) | <0.001 |
| cGFR | | 1.33 (1.10-1.51) | 0.014 | 1.12 (1.03-1.39) | 0.036 | | 1.35 (1.21-1.45) | 0.039 | 1.22 (1.12-1.43) | 0.053 |
| Blood glu. | | 3.32 (1.79-5.87) | <0.001 | 2.62 (1.48-4.62) | <0.001 | | 4.06 (3.55-4.78) | <0.001 | 4.06 (3.55-4.78) | <0.001 |
| AgeDiff | | 3.16 (2.11-5.28) | <0.001 | 2.74 (1.69-3.88) | <0.001 | | 3.32 (2.37-4.14) | <0.001 | 2.45 (1.76-3.40) | <0.001 |
| | CKD | | | | | Hypertension | | | | |
| Sex | | 0.71 (0.53-0.93) | 0.003 | 0.69 (0.64-0.72) | 0.007 | | 0.93 (0.73-1.03) | <0.001 | 0.91 (0.75-1.02) | <0.001 |
| BMI | | 1.04 (1.03-1.00) | <0.001 | 1.03 (1.02-1.06) | <0.001 | | 1.21 (1.03-1.34) | <0.001 | 1.11 (1.04-1.21) | <0.001 |
| Height | | 0.96 (0.93-0.99) | 0.007 | 1.01 (1.00-1.03) | 0.010 | | 0.74 (0.55-0.91) | 0.013 | 0.73 (0.66-0.75) | 0.033 |
| Weight | | 1.06 (1.03-1.08) | 0.014 | 1.00 (1.00-1.01) | 0.005 | | 1.26 (1.00-1.52) | <0.001 | 1.18 (1.13-1.31) | <0.001 |
| Smoking | | 1.44 (1.15-1.61) | <0.001 | 1.32 (1.19-1.52) | <0.001 | | 1.74 (1.45-1.91) | <0.001 | 1.62 (1.39-1.72) | <0.001 |
| SBP | | 1.55 (1.05-1.83) | <0.001 | 1.39 (1.02-1.43) | <0.001 | | 4.63 (2.45-6.48) | <0.001 | 4.31 (2.55-5.98) | <0.001 |
| DBP | | 1.47 (1.13-1.62) | 0.005 | 1.36 (1.15-1.55) | 0.023 | | 3.28 (2.11-5.47) | <0.001 | 3.08 (2.33-4.84) | <0.001 |
| cGFR | | 3.16 (2.60-3.51) | <0.001 | 3.37 (2.85-3.64) | <0.001 | | 1.21 (1.13-1.34) | 0.016 | 1.22 (1.12-1.43) | 0.062 |
| Blood glu. | | 1.21 (0.99-1.31) | <0.001 | 1.07 (1.04-1.11) | <0.001 | | 1.03 (1.00-1.05) | 0.014 | 1.02 (1.00-1.06) | 0.031 |
| AgeDiff | | 4.00 (3.55-4.78) | <0.001 | 4.14 (3.49-4.51) | <0.001 | | 3.22 (2.46-3.76) | <0.001 | 3.11 (2.12-3.52) | <0.001 |
| | CVD | | | | | Stroke | | | | |
| Sex | | 0.82 (0.62-0.94) | 0.005 | 0.71 (0.63-0.77) | 0.011 | | 1.02 (0.99-1.09) | 0.005 | 0.71 (1.00-1.06) | 0.011 |
| BMI | | 1.24 (1.06-1.31) | 0.004 | 1.14 (1.04-1.21) | 0.014 | | 1.03 (1.01-1.04) | 0.003 | 1.03 (1.00-1.05) | 0.012 |
| Height | | 0.93 (0.88-0.97) | 0.063 | 0.92 (0.91-0.99) | 0.085 | | 1.01 (1.00-1.03) | 0.024 | 1.02 (1.00-1.04) | 0.035 |
| Weight | | 1.31 (1.05-1.48) | 0.014 | 1.24 (1.01-1.42) | 0.033 | | 1.04 (1.00-1.08) | 0.014 | 1.03 (1.01-1.06) | 0.033 |
| Smoking | | 1.84 (1.35-2.32) | <0.001 | 1.32 (1.19-1.52) | <0.001 | | 1.54 (1.32-1.77) | <0.001 | 1.51 (1.30-1.04) | <0.001 |
| SBP | | 1.55 (1.05-1.83) | 0.024 | 1.29 (1.02-1.43) | 0.035 | | 1.45 (1.23-1.71) | 0.014 | 1.29 (1.12-1.44) | 0.023 |
| DBP | | 1.47 (1.13-1.62) | 0.017 | 1.36 (1.15-1.55) | 0.043 | | 1.11 (1.05-1.20) | 0.014 | 1.36 (1.15-1.55) | 0.043 |
| cGFR | | 1.16 (1.01-1.31) | <0.001 | 1.09 (1.01-1.29) | <0.001 | | 1.32 (1.21-1.44) | <0.001 | 1.15 (1.08-1.23) | <0.001 |
| Blood glu. | | 2.52 (1.49-3.72) | <0.001 | 2.02 (1.68-3.01) | <0.001 | | 2.13 (1.74-2.94) | <0.001 | 2.06 (1.74-2.64) | <0.001 |
| AgeDiff | | 3.76 (2.25-4.93) | <0.001 | 3.14 (1.99-4.33) | <0.001 | | 3.06 (2.25-4.93) | <0.001 | 3.14 (1.99-4.33) | <0.001 |

### REFERENCE

[1] L. Jia, W. Zhang, and X. Chen, ‘Common methods of biological age estimation’, Clin. Interv. Aging, vol. 12, p. 759, 2017.
[2] M. R. Hamczyk, R. M. Nevado, A. Barettino, V. Fuster, and V. Andres, ‘Biological versus chronological aging: JACC focus seminar’, J Am. Coll. Cardiol., vol. 75, no. 8, pp. 919-930, 2020.
[3] A. Vaiserman and D. Krasnienkov, ‘Telomere length as a marker of biological age: state-of-the-art, open issues, and future perspectives’, Front. Genet., vol. 11, p. 630186, 2021.
[4] G. Hannum et al., ‘Genome-wide methylation profiles reveal quantitative views of human aging rates’, Mol. Cell, vol. 49, no. 2, pp. 359-367, 2013.
[5] J. H. Cole et al., ‘Brain age predicts mortality’, Mol. Psychiatry, vol. 23, no. 5, pp. 1385-1392, 2018.
[6] J. Wang et al., ‘Gray matter age prediction as a biomarker for risk of dementia’, Proc. Natl. Acad. Sci., vol. 116, no. 42, pp. 21213-21218, 2019.
[7] Z. Zhu et al., ‘Retinal age gap as a predictive biomarker for mortality risk’, Br. J. Ophthalmol., 2022.
[8] C. Liu et al., ‘Biological age estimated from retinal imaging: a novel biomarker of aging’, in International Conference on Medical Image Computing and Computer-Assisted Intervention, Springer, 2019, pp. 138-146.
[9] X. Xia et al., ‘Three-dimensional facial-image analysis to predict heterogeneity of the human ageing rate and the impact of lifestyle’, Nat. Metab., vol. 2, no. 9, pp. 946-957, 2020.
[10] W. Chen et al., ‘Three-dimensional human facial morphologies as robust aging markers’, Cell Res., vol. 25, no. 5, pp. 574-587, 2015.
[11] A. London, I. Benhar, and M. Schwartz, ‘The retina as a window to the brain—from eye research to CNS disorders’, Nat. Rev. Neurol., vol. 9, no. 1, pp. 44-53, 2013.
[12] C. Y Cheung et al., ‘Deep-learning retinal vessel calibre measurements and risk of cognitive decline and dementia’, Brain Commun., vol. 4, no. 4, p. fcac212, 2022.
[13] W. Hu et al., ‘Retinal age gap as a predictive biomarker of future risk of Parkinson's disease’, Age Ageing, vol. 51, no. 3, p. afac062, 2022.
[14] Y. Li, J. Cui, Y. Liu, K. Chen, L. Huang, and Y. Liu, ‘Oral, tongue-coating microbiota, and metabolic disorders: a novel area of interactive research’, Front. Cardiovasc. Med. , p. 922, 2021.
[15] C. Lu et al., ‘Oral-Gut Microbiome Analysis in Patients With Metabolic-Associated Fatty Liver Disease Having Different Tongue Image Feature’, Front. Cell. Infect. Microbiol., p. 341, 2022.
[16] S. E. Kjeldsen, ‘Hypertension and cardiovascular risk: General aspects’, Pharmacol. Res., vol. 129, pp. 95-99, 2018.
[17] I. H. De Boer et al., ‘Diabetes and hypertension: a position statement by the American Diabetes Association’, Diabetes Care, vol. 40, no. 9, pp. 1273-1284, 2017.
[18] G. Ke et al., ‘Lightgbm: A highly efficient gradient boosting decision tree’, Adv. Neural Inf Process. Syst., vol. 30, 2017.
[19] S. M. Lundberg and S.-I. Lee, ‘A unified approach to interpreting model predictions’, Adv. NeuralInf Process. Syst., vol. 30, 2017.
[20] S. Horvath, ‘DNA methylation age of human tissues and cell types’, Genome Biol., vol. 14, no. 10, pp. 1-20, 2013.
[21] J. H. Cole and K. Franke, ‘Predicting age using neuroimaging: innovative brain ageing biomarkers’, Trends Neurosci., vol. 40, no. 12, pp. 681-690, 2017.
[22] M. J. Peters et al., ‘The transcriptional landscape of age in human peripheral blood’, Nat. Commun., vol. 6, no. 1, pp. 1-14, 2015.
[23] C. I. Weidner et al., ‘Aging of blood can be tracked by DNA methylation changes at just three CpG sites’, Genome Biol., vol. 15, no. 2, pp. 1-12, 2014.
[24] J. G. Fleischer et al., ‘Predicting age from the transcriptome of human dermal fibroblasts’, Genome Biol., vol. 19, no. 1, pp. 1-8, 2018.
[25] E. Putin et al., ‘Deep biomarkers of human aging: application of deep neural networks to biomarker development’, Aging, vol. 8, no. 5, p. 1021, 2016.
[26] P. Mamoshina et al., ‘Population specific biomarkers of human aging: a big data study using South Korean, Canadian, and Eastern European patient populations’, J. Gerontol. Ser. A, vol. 73, no. 11, pp. 1482-1490, 2018.
[27] C. Y. Cheung, M. K. Ikram, C. Chen, and T. Y. Wong, ‘Imaging retina to study dementia and stroke’, Prog. Retin. Eye Res., vol. 57, pp. 89-107, 2017.
[28] N. Patton, T. Aslam, T. MacGillivray, A. Pattie, I. J. Deary, and B. Dhillon, ‘Retinal vascular image analysis as a potential screening tool for cerebrovascular disease: a rationale based on homology between cerebral and retinal microvasculatures’, J. Anat., vol. 206, no. 4, pp. 319-348, 2005.
[29] J. Cavanagh and H. Jones, ‘Glycogenosomes in the aging rat brain: their occurrence in the visual pathways’, Acta Neuropathol. (Berl.), vol. 99, no. 5, pp. 496-502, 2000.
[30] P.-C. Hsu et al., ‘Gender-and age-dependent tongue features in a community-based population’, Medicine (Baltimore), vol. 98, no. 51, 2019.
[31] R. B. Shaw Jr et al., ‘Aging of the facial skeleton: aesthetic implications and rejuvenation strategies’, Plast. Reconstr. Surg., vol. 127, no. 1, pp. 374-383, 2011.
[32] S. Kumar, E.-H. Wang, M. J. Pokabla, and R. J. Noecker, ‘Teleophthalmology assessment of diabetic retinopathy fundus images: smartphone versus standard office computer workstation’, Telemed. E-Health, vol. 18, no. 2, pp. 158-162, 2012.
[33] Y Wang et al., ‘China suboptimal health cohort study: rationale, design and baseline characteristics’, J Transl. Med., vol. 14, no. 1, pp. 1-12, 2016.
[34] N. Otsu, ‘A threshold selection method from gray-level histograms’, IEEE Trans. Syst. Man Cybern., vol. 9, no. 1, pp. 62-66, 1979.
[35] J. Ning, L. Zhang, D. Zhang, and C. Wu, ‘Interactive image segmentation by maximal similarity based region merging’, Pattern Recognit., vol. 43, no. 2, pp. 445-456, 2010.
[36] J. A. Sethian, ‘A fast marching level set method for monotonically advancing fronts.’, Proc. Natl. Acad. Sci., vol. 93, no. 4, pp. 1591-1595, 1996.
[37] N. Sebkhi, N. Santus, A. Bhavsar, S. Siahpoushan, and O. T. Inan, ‘Evaluation of a Wireless Tongue Tracking System on the Identification of Phoneme Landmarks’, IEEE Trans. Biomed. Eng., vol. 68, no. 4, pp. 1190-1197, 2020.
[38] Z. Liu et al., ‘Swin transformer v2: Scaling up capacity and resolution’, in Proceedings of the IEEE/CVF Conference on Computer Vision and Pattern Recognition, 2022, pp. 12009-12019.
[39] I. Loshchilov and F. Hutter, ‘Decoupled weight decay regularization’, ArXiv Prepr. ArXiv171105101, 2017.
[40] A. Paszke et al., ‘Pytorch: An imperative style, high-performance deep learning library’, Adv. Neural Inf Process. Syst., vol. 32, 2019.
[41] I. Loshchilov and F. Hutter, ‘Sgdr: Stochastic gradient descent with warm restarts’, ArXiv Prepr. ArXiv160803983, 2016.
[42] H. Zhang, M. Cisse, Y. N. Dauphin, and D. Lopez-Paz, ‘mixup: Beyond empirical risk minimization’, ArXiv Prepr. ArXiv171009412, 2017.
[43] E. D. Cubuk, B. Zoph, J. Shlens, and Q. V. Le, ‘Randaugment: Practical automated data augmentation with a reduced search space’, in Proceedings of the IEEE CVF conference on computer vision and pattern recognition workshops, 2020, pp. 702-703.
[44] A. Chattopadhay, A. Sarkar, P. Howlader, and V. N. Balasubramanian, ‘Grad-cam++: Generalized gradient-based visual explanations for deep convolutional networks’, in 2018 IEEE winter conference on applications of computer vision (WACV), IEEE, 2018, pp. 839-847.
[45] D. Giavarina, ‘Understanding bland altman analysis’, Biochem. Medica, vol. 25, no. 2, pp. 141-151, 2015.
[46] N. E. Breslow, ‘Statistical methods in cancer research II. The design and analysis of cohort studies’, IARC Sci. Publ., vol. 82, pp. 1-406, 1987.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method of training a model for biological age estimation, wherein the model comprises a first projection module, a second projection module, a third projection module, and a multimodal transformer comprising a cross-attention module, the method comprising:
(a) obtaining multimodal input data of a subject, wherein the multimodal input data comprise data in a first modality, data in a second modality, and data in a third modality:
(b) passing the data in a first modality, the data in a second modality, and the data in a third modality to the first projection module, the second projection module, and the third projection module, respectively, to construct the corresponding image tokens and classification tokens:
(c) passing the image tokens and classification tokens to the multimodal transformer comprising the cross-attention module, wherein the cross-attention module comprises three branches, wherein each branch processes image tokens of one of the three modalities, and wherein the cross-attention module fuses the image tokens and the classification tokens using cross-attention fusion comprising fusing a classification token from one of three modalities and image tokens from the other two modalities; and
(d) passing an output of the multimodal transformer to a plurality of multilayer perceptrons for biological age estimation, thereby training the model for providing biological age estimation.

2. The method of claim 1, wherein each of the first projection module, the second projection module, and the third projection module is independently a linear projection module.

3. The method of claim 1, wherein the first projection module, the second projection module, and the third projection module are linear projection modules.

4. The method of claim 1, wherein the multimodal transformer comprises a first Swin-Transformer encoder for the image tokens and classification tokens from the data in the first modality, a second Swin-Transformer encoder for the image tokens and classification tokens from the data in the second modality, and a third Swin-Transformer encoder for the image tokens and classification tokens from the data in the third modality.

5. The method of claim 1, wherein the multimodal transformer comprises Z-stack encoders each having a cross-attention module.

6. The method of claim 5, wherein the cross-attention module in each stack comprises three branches, each of which is configured to process image tokens of one of the three modalities.

7. The method of claim 1, wherein the first modality, the second modality, and the third modality are medical image modalities.

8. The method of claim 1, wherein the first modality, the second modality, and the third modality are retinal images, tongue images, and facial images, respectively.

9. The method of claim 8, wherein the retinal images are fundus images.

10. The method of claim 8, wherein the facial images are 3D facial stereophotogrammetry images.

11. The method of claim 1, further comprising obtaining the difference AgeDiff between an estimated biological age (BA) of an individual and the individual's chronological age (CA), wherein AgeDiff=|BA−CA|.

12. A method of for biological age estimation in an individual, the method comprising:
receiving a prompt for obtaining an estimated biological age and data in the first modality, data in the second modality, and data in the third modality of the individual, and
generating the estimated biological age by inputting the prompt and the data in the three modalities in a trained model generated by the method of claim 1.

13. The method of claim 12, further comprising obtaining the difference AgeDiff between the estimated biological age (BA) of the individual and the individual's chronological age (CA), wherein AgeDiff=|BA−CA|.

14. The method of claim 13, comprising using AgeDiff to predict a 5-year risk of the individual developing a chronic disease.

15. The method of claim 13, comprising using a combination of AgeDiff and one or more known risk factors for a chronic disease to predict a 5-year risk of the individual developing the chronic disease.

16. The method of claim 14, wherein the chronic disease is coronary heart disease (CHD), cardiovascular disease (CVD), chronic kidney disease (CKD), stroke, hypertension, or diabetes.

17. A method of training a model for biological age estimation, wherein the model comprises a first projection module, a second projection module, a third projection module, and a multimodal transformer comprising a cross-attention module, the method comprising:
(a) obtaining multimodal input data of a subject, wherein the multimodal input data comprise three image modalities: retinal images, tongue images, and facial images:
(b) passing the retinal images, tongue images, and facial images to the first projection module, the second projection module, and the third projection module, respectively, to construct the corresponding image tokens and classification tokens;
(c) passing the image tokens and classification tokens to the multimodal transformer comprising the cross-attention module,
wherein the cross-attention module comprises three branches that process image tokens of the retinal images, the tongue images, and facial images, respectively, and
wherein the cross-attention module fuses the image tokens and the classification tokens using cross-attention fusion comprising fusing classification tokens from one of the three image modalities and image tokens from the other two image modalities:
(d) passing an output of the multimodal transformer to a plurality of multilayer perceptrons for biological age estimation, thereby training the model for providing biological age estimation; and
(e) obtaining the difference AgeDiff between the estimated biological age (BA) of an individual and the individual's chronological age (CA), wherein AgeDiff=|BA−CA|.

18. A system comprising:
at least one hardware processor; and
one or more software modules configured to, when executed by the at least one hardware processor, perform the method of claim 1.

19. A non-transitory computer-readable medium having instructions stored thereon, wherein the instructions, when executed by a processor, cause the processor to perform the method of claim 1.

20. A system comprising:
at least one hardware processor,
non-transitory computer-readable medium coupled to at least one hardware processor, optionally wherein the coupling is over a network; and
instructions stored in the non-transitory computer-readable medium, wherein the instructions when implemented by the processor, configure the system to perform the method of claim 1.
